(19) United States
(12) Patent Application Publication
(10) Pub. No.: US 2024/0085326 A1
(43) Pub. Date: Mar. 14, 2024

(54) METHOD, COMPUTER PROGRAM AND DATA PROCESSING UNIT FOR PREPARING OBSERVATION OF FLUORESCENCE INTENSITY, METHOD FOR OBSERVING FLUORESCENCE INTENSITY, AND OPTICAL OBSERVATION SYSTEM

(71) Applicant: Carl Zeiss Meditec AG, Jena (DE)

(72) Inventors: Alois Regensburger, Oberkochen (DE); Christoph Hauger, Oberkochen (DE)

(21) Appl. No.: 18/238,445

(22) Filed: Aug. 25, 2023

(30) Foreign Application Priority Data
Aug. 25, 2022 (DE) 10 2022 121 504.0
Aug. 25, 2022 (DE) 10 2022 121 505.9

(51) Int. Cl. G01N 21/64; G02B 21/00

(52) U.S. Cl. CPC G01N 21/6428; G01N 21/6458; G02B 21/0012; G01N 2021/6439; G01N 2021/6478; G01N 2201/129

(57) ABSTRACT

What is provided is a method for preparing the observation of a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100) using which fluorescence radiation is able to be observed, provided that this has a certain minimum intensity. The method comprises the following steps:
determining the parameter value of at least one parameter which influences the observation of the fluorescence intensity, and
simulating the fluorescence intensity expected for the respective object regions (3A-H) on the basis of the determined parameter value of the at least one parameter and a model of the influence of the at least one parameter on the fluorescence intensity.

Within the scope of the simulation, a minimum concentration of the fluorescent dye is predefined and the fluorescence intensity expected with the minimum concentration is determined for each object region (3A-H) based on the simulation.

Also provided are a computer program, a computer-implemented method, a data processing unit (6) and an optical observation system (100) for preparing the observation of a fluorescence intensity of fluorescence radiation of a fluorescent dye in an observation object (3).

### CROSS-REFERENCE TO RELATED APPLICATIONS

This application claims the priority of the German patent application Nos. 10 2022 121 505.9 and 10 2022 121 504.0, filed on Aug. 25, 2022. The entire contents of both applications are incorporated herein by reference.

### DESCRIPTION

The present invention relates to a method for preparing the observation of a fluorescence intensity of fluorescence radiation of a fluorescent dye in an observation object, and to a method for observing a fluorescence intensity of fluorescence radiation. In addition, the invention relates to a optical observation system. Also provided are a computer program, a computer-implemented method and a data processing unit for preparing the observation of a fluorescence intensity of fluorescence radiation of a fluorescent dye in an observation object.

Various fluorescence options enabling the observation of fluorescence may be integrated in modern optical observation systems, which may comprise optical observation devices such as surgical microscopes and endoscopes for example. If the intention is to carry out a fluorescence measurement, then the latter is frequently based purely on the color impression and the perceived brightness under observation with the eye. However, this color impression and the brightness depend very strongly on very different parameters during the recording, and this does not allow for a reliable quantitative measurement. There therefore are recommendations to set the working distance and the illumination brightness to specific values in order to make fluorescence measurements better comparable.

For example, fluorescence recordings within the scope of neurosurgical operations depend on a multiplicity of parameters which influence the perceivable fluorescence intensity. In this context, it is frequently necessary to repeatedly modify parameters, for instance the working distance, the zoom setting, etc., during the neurosurgical operation. However, the observed or measured fluorescence intensity also changes with each modification. This leads to a tumor marked by a fluorescent dye shining brighter and darker at different times, depending on the current parameter set. However, since the luminosity of the fluorescence is included in the diagnosis for at least some fluorescence methods, comparability between a plurality of fluorescence measurements in different surgical situations, made by different users, and carried out in different clinics is sought after. In particular, quantitative fluorescence measurement methods are also sought after.

Quantitative measurements are possible by means of contact measurements at tissue points; however, such punctiform measurements made by handheld contact devices are not practical for the visualization of the fluorescence over a relatively large area or even live during the resection. By way of example, such contact measurements are described in “Quantitative fluorescence in intracranial tumor: implications for ALA-induced PpIX as an intraoperative biomarker”, Roberts et al, Neurosurg. 2011 July; 115(1): 11-17. doi:10.3171/2011.2.JNS101451.

US 2019/227288 A1 describes a method for normalizing fluorescence intensities. In the method, parameter values of the observation beam path in an optical observation device, in particular the values for the settings of magnification and working distance, are acquired. The acquired parameter values and the influence of the corresponding parameters on the fluorescence intensity are used to set an exposure parameter for the image recording, in such a way that the influence of a modified magnification or a modified working distance on the recorded fluorescence intensities is compensated.

Fluorescence images are recorded in US 2016/278678 A1 and corrected on the basis of a 3-D surface model. In particular, fluorescence images allowing a quantification of superficial and surface-near dyes are recorded. In the process, image deformations based on settings of the image recording and on the surface orientation of the observation object are determined and taken into account by way of a suitable image distortion.

Although quantitative fluorescence measurements are possible using the method described in US 2019/227288 A1 and US 2016/278678 A1, for example, there is also the requirement here that the fluorescence intensity is sufficient to be able to be detected. There is therefore the possibility of the quantitative measurement failing despite a suitable quantitative fluorescence measurement method, since for example, following a change in zoom level or working distance, the fluorescence intensity is no longer sufficient to be able to be detected for example by an image sensor that is used.

The object of the invention is therefore to provide a method, a computer program and a data processing unit for preparing the observation of a fluorescence intensity of fluorescence radiation and a method for observing a fluorescence intensity and an optical observation system, using all of which the likelihood of failure in a fluorescence measurement is able to be reduced.

This object is achieved according to the invention by a method for preparing the observation of a fluorescence intensity as claimed in claim 1, by a method for observing a fluorescence intensity as claimed in claim 11, by an optical observation system as claimed in claim 12, by a computer-implemented method as claimed in claim 20, by a computer program as claimed in claim 21 and a data processing unit as claimed in claim 22. The dependent claims contain advantageous configurations of the invention.

According to the invention, a method for preparing the observation of a fluorescence intensity of fluorescence radiation of a fluorescent dye in an observation object is provided. The observation object, which may also be a region of interest of a larger object, has object regions that differ from one another in terms of their depth and/or their orientation. The fluorescence intensity is intended to be observed using an optical observation system using which it is possible to observe fluorescence radiation, provided that this has a certain minimum intensity. The minimum intensity may in this case be defined by the sensitivity of an image sensor used in the optical observation system. The image sensor may for example be part of an optical observation device used in the optical observation system, such as for instance a camera, a surgical microscope, an endoscope, etc. Cameras, surgical microscopes, endoscopes, etc. are examples of optical observation devices in which the imaging is implemented by means of an imaging beam path. Optical observation devices in which the imaging is implemented by means of an imaging beam path should be distinguished from those optical observation devices in which imaging is implemented by virtue of scan points in the object being scanned and intensities recorded at scan points subsequently being combined to form an image. An imaging beam path is not present in that context. Examples of such optical observation devices include laser scanning microscopes or OCT systems (OCT: Optical Coherence Tomography). In the case of purely visual observation of the fluorescence with the optical observation device that is used as well, however, it is necessary to achieve a certain minimum intensity of the fluorescence radiation so that the eye is able to perceive it. The method comprises the following steps:
determining the parameter value of at least one parameter which influences the observation of the fluorescence intensity. By way of example, the parameter value may be determined here by acquiring the parameter value by means of a suitable sensor or by retrieving the parameter value from a controller. However, there is also the option of determining the parameter value by virtue of calculating the latter from at least one other acquired or retrieved parameter value on the basis of a model.
simulating the fluorescence intensity expected for the respective object regions on the basis of the determined parameter value of the at least one parameter and a model of the influence of the at least one parameter on the fluorescence intensity.

The method according to the invention is distinguished in that a minimum concentration of the fluorescent dye is predefined within the scope of the simulation and the fluorescence intensity expected with the minimum concentration is determined for each object region based on the simulation.

Based on the simulation of the fluorescence intensity expected with the determined parameter values for the minimum concentration of the fluorescent dye, it is possible to determine beforehand which regions of the observation object, with the currently set parameters, make it possible to detect the minimum concentration of the fluorescent dye. This may be used to check whether the determined expected fluorescence intensity is sufficient to be able to be detected by the optical observation system with the given sensitivity thereof. The check makes it possible to prevent carrying out fluorescence marking that does not enable the detection of the minimum concentration of the fluorescent dye at all in the regions of interest of the observation object. Wasting of time caused by non-expedient fluorescence measurements during an operation may thereby be avoided. To prevent a non-expedient fluorescence measurement, an optical and/or acoustic alert may for example be output when a check reveals that the expected fluorescence intensity determined for the minimum concentration of the fluorescent dye is not sufficient, in each object region, to be able to be detected by the optical observation system with the given sensitivity thereof.

A graphical display may advantageously be generated, which displays the object regions in which the expected fluorescence intensity determined for the minimum concentration of the fluorescent dye is sufficient to be able to be detected by the optical observation system with the given sensitivity thereof. The user is thereby given the ability to decide whether the expected fluorescence intensity determined for the minimum concentration of the fluorescent dye is sufficient, in the object regions relevant to them, to be able to carry out the fluorescence observation in an expedient manner.

The method additionally also offers the possibility, based on the simulation, of determining an improved parameter value, in particular an optimized parameter value, for the at least one parameter such that the expected fluorescence intensity simulated with the optimized parameter value for the minimum concentration of the fluorescent dye is sufficient, in as many object regions as possible, in particular where possible in all object regions, to be able to be detected by the optical observation system with the given sensitivity thereof. The improved or optimized parameter value for the at least one parameter may then for example be displayed in order to be set manually or output to an automatic setting unit that sets the current parameter value of the at least one parameter automatically to the improved or optimized parameter value. It is thereby possible to reduce the proportion of non-expedient fluorescence measurements to those fluorescence measurements in which it is not possible to improve the parameter value of the at least one parameter value.

Information about the depth distribution of the object regions and/or information about the orientation of the object regions may in particular be used within the scope of the simulation. Said one or more items of information may for example be present in the form of a 3-D model of the observation object, in the form of a depth map of the observation object, etc. This information makes it possible, within the scope of the simulation, to take into consideration the dependency of the fluorescence intensity on the distance of the illumination system and/or of the optical observation system from the respective object regions and/or the orientation of the illumination system and/or of the optical observation system in relation to the respective object regions. It becomes possible in particular also to take into consideration shading effects.

At least the parameter value of one of the following parameters may be determined as the at least one parameter value and taken into consideration in the simulation:
distance of an optical observation device of the optical observation system from the object regions. This distance may be determined for example based on the information about the depth distribution of the object regions and the information about the orientation of the object regions, and the position and orientation of the optical observation device in relation to the observation object. By way of example, a navigation system, stereography, etc. may be used to determine the position and orientation of the optical observation device in relation to the observation object.
orientation of the optical observation device in relation to the object regions. The orientation, like the distance, may be determined for example based on the information about the depth distribution of the object regions and the information about the orientation of the object regions, and the position and orientation of the optical observation device in relation to the observation object.
zoom setting of the optical observation device.
front focal distance of the optical observation device.
stop setting of the optical observation device.
gain of an image sensor used in the optical observation device.
exposure duration of an image sensor used in the optical observation system.
nonlinearities of an image sensor used in the optical observation system.
distance of an illumination system from the object regions. This distance may be determined for example based on the information about the depth distribution of the object regions and the information about the orientation of the object regions, and the position and orientation of the illumination system in relation to the observation object. By way of example, a navigation system, stereography, etc. may be used to determine the position and orientation of the illumination system in relation to the observation object.
orientation of an illumination system in relation to the object regions. The orientation, like the distance, may be determined for example based on the information about the depth distribution of the object regions and the information about the orientation of the object regions, and the position and orientation of the illumination system in relation to the observation object.
intensity of an illumination light source.
spectral intensity distribution of an illumination light source.
zoom setting of an illumination zoom.
position of an illumination stop.

All of said parameters influence the fluorescence intensity that is able to be detected in the presence of the minimum concentration of fluorescent dye, for example by the eye or an image sensor of the optical observation device. The more these parameters are taken into consideration in the simulation, the more accurate the simulation. In practice, the available computer capacity and/or the configuration of the observation system may limit the number of parameters able to be used to the parameters that are needed or most important within the scope of the intended fluorescence observation.

A light source for fluorescence excitation, typically a xenon lamp or an LED, is subject to ageing effects that lead to the spectral intensity distribution of the illumination source changing over time. It is therefore advantageous when the spectral intensity distribution of the illumination light source is determined based on the value of a service life counter of the illumination source and its nominally set intensity using a degradation model of the illumination source.

In one advantageous development of the method according to the invention for preparing the observation of a fluorescence intensity, at least one reference measurement with a reference concentration of the fluorescent dye is carried out using a reference parameter value for the at least one parameter which influences the observation of the fluorescence intensity, in order to obtain a reference value for the fluorescence intensity at the reference concentration of the fluorescent dye.

A simulation of the fluorescence intensity expected for the respective object regions is then carried out, in which a change in the fluorescence intensity in comparison to the reference intensity is determined for a deviation of the parameter value of the at least one parameter which influences the observation of the fluorescence intensity from the reference parameter value.

Finally, a compensation factor is determined, by means of which it is possible to compensate for a change in the fluorescence intensity in a digital image which is caused by the deviation of the parameter value of the at least one parameter which influences the observation of the fluorescence intensity from the reference parameter value.

The reference measurement and the compensation factor make it possible to depict surface regions with the same concentration of fluorescent dye with the same intensity of the fluorescence radiation, independently of the distance and the orientation of a surface region in relation to the illumination system of the optical observation system and in relation to the optical observation device of the optical observation system.

Advantageously, the at least one reference measurement is carried out with reference parameter values for each parameter which influences the observation of the fluorescence intensity and which is of relevance subsequently when observing a fluorescence intensity. The reference measurement supplies more accurate results in this way. By way of example, the reference measurement may be carried out on a reference observation object with a known surface geometry, in particular a plane surface, with the optical observation device of the optical observation system and the illumination system of the optical observation system each being in a reference position and a reference orientation in relation to the reference observation object. Within the scope of the described development, it is also possible to carry out a plurality of reference measurements with a plurality of mutually different reference concentrations, in order to obtain reference values for the fluorescence intensities assigned to the various reference concentrations. As a result, the reference intensity exhibiting the smallest deviation from the captured fluorescence intensity may be used in each case for the calculation of the compensation factors. Overall, the accuracy of the calculated compensation factors may be increased.

According to a second aspect of the invention, a method for observing a fluorescence intensity of fluorescence radiation of a fluorescent dye in an observation object is provided. The observation object, which may also be a region of interest of a larger object, has object regions that differ from one another in terms of their depth and/or their orientation. The fluorescence intensity is observed using an optical observation system using which it is possible to observe fluorescence radiation, provided that this has a certain minimum intensity. The minimum intensity may in this case be defined by the sensitivity of an image sensor used in the optical observation system. The image sensor may in this case be part of an optical observation device used in the optical observation system, for instance a camera, a surgical microscope, an endoscope, etc. In the case of purely visual observation of the fluorescence as well, however, it is necessary to achieve a certain minimum intensity of the fluorescence radiation so that the eye is able to perceive it. The method according to the invention for preparing the observation of a fluorescence intensity of fluorescence radiation of a fluorescent dye in an observation object, or one of its advantageous developments, is used within the scope of the method for observing a fluorescence intensity.

Using the method for preparing the observation of a fluorescence intensity of fluorescence radiation of a fluorescent dye, it is possible to determine beforehand which regions of the observation object, with the currently set parameters, make it possible to detect the minimum concentration of the fluorescent dye. It is thereby possible to assess, before the observation of the fluorescence intensity, whether the measurement makes sense. If the measurement does not make sense, the parameter value of the at least one parameter which influences the observation of the fluorescence intensity may be improved or optimized with a view to being able to achieve a fluorescence intensity able to be detected using the optical observation system in as many surface regions as possible with the minimum concentration of fluorescent dye. The improved or optimized parameter value of the at least one parameter which influences the observation of the fluorescence intensity may for example contain at least one of the following changes in relation to the original parameter value: A changed intensity of the illumination radiation, a changed position of the optical observation device used in the optical observation system and/or of the illumination system used in the optical observation system in relation to the observation object, a changed orientation of the optical observation device used in the optical observation system and/or of the illumination system used in the optical observation system in relation to the observation object, a changed zoom setting of the optical observation device used in the optical observation system and/or of the illumination system used in the optical observation system, etc.

According to a third aspect of the invention, an optical observation system is provided, enabling the observation of a fluorescence intensity of fluorescence radiation of a fluorescent dye in an observation object that comprises object regions that differ from one another in terms of their depth and/or their orientation, provided that this has a certain minimum intensity. The minimum intensity may in this case be defined by the sensitivity of an image sensor used in the optical observation system. In the case of purely visual observation of the fluorescence as well, however, it is necessary to achieve a certain minimum intensity of the fluorescence radiation so that the eye is able to perceive it. The optical observation system may comprise for example a camera, a surgical microscope, an endoscope, etc. as an observation device. Cameras, surgical microscopes, endoscopes, etc. are examples of optical observation devices in which the imaging is implemented by means of an imaging beam path. Optical observation devices in which the imaging is implemented by means of an imaging beam path should be distinguished from those optical observation devices in which imaging is implemented by virtue of scan points in the object being scanned and intensities recorded at scan points subsequently being combined to form an image. An imaging beam path is not present in that context. Examples of such optical observation devices include laser scanning microscopes or OCT systems (OCT: Optical Coherence Tomography).

The optical observation system comprises a determination device for determining the parameter value of at least one parameter which influences the observation of the fluorescence intensity and a simulation device for simulating the fluorescence intensity expected for the respective object regions on the basis of the determined parameter value of the at least one parameter and a model of the influence of the at least one parameter on the fluorescence intensity. By way of example, the parameter value may be determined here by acquiring the parameter value by means of a suitable sensor or by retrieving the parameter value from a controller. However, there is also the option of determining the parameter value by virtue of calculating the latter from at least one other acquired or retrieved parameter value on the basis of a model. The optical observation system furthermore comprises an evaluation device that is designed, for a minimum concentration of the fluorescent dye that is predefined within the scope of the simulation, to determine the fluorescence intensity expected with the minimum concentration for each object region based on the simulation.

The optical observation system makes it possible to implement the method for observing a fluorescence intensity of fluorescence radiation of a fluorescent dye including the method for preparing the observation of a fluorescence intensity of fluorescence radiation of a fluorescent dye, such that the optical observation system may be used to achieve the advantages described in relation to these two methods.

In advantageous developments of the optical observation system, this is designed to implement the advantageous developments of the method according to the invention for preparing the observation of a fluorescence intensity of fluorescence radiation of a fluorescent dye, such that the advantages described in relation to the advantageous developments of the method according to the invention for preparing the observation of a fluorescence intensity of fluorescence radiation of a fluorescent dye may be achieved with the optical observation system. The advantages achieved through the advantageous developments of the optical observation system are therefore not described again. Reference is made instead to the description of the advantages of the developments of the method according to the invention for preparing the observation of a fluorescence intensity of fluorescence radiation of a fluorescent dye.

In a first of the advantageous developments, the evaluation device is designed to carry out a check, for each object region, as to whether the determined expected fluorescence intensity is sufficient to be able to be detected by the optical observation system with the given sensitivity thereof.

In a second of the advantageous developments, the simulation device is designed to use information about the depth distribution of the object regions and/or information about the orientation of the object regions within the scope of the simulation.

In a third of the advantageous developments, the determination device is designed to determine at least the parameter value of one of the following parameters, and the simulation device is designed to take this at least one parameter value into consideration in the simulation:
distance of an optical observation device of the optical observation system from the object regions. This distance may be determined for example based on the information about the depth distribution of the object regions and the information about the orientation of the object regions, and the position and orientation of the optical observation device in relation to the observation object. By way of example, a navigation system, stereography, etc. may be used to determine the position and orientation of the optical observation device in relation to the observation object.
orientation of the optical observation device in relation to the object regions. The orientation, like the distance, may be determined for example based on the information about the depth distribution of the object regions and the information about the orientation of the object regions, and the position and orientation of the optical observation device in relation to the observation object.
zoom setting of the optical observation device.
front focal distance of the optical observation device.
stop setting of the optical observation device.
gain of an image sensor used in the optical observation device.
exposure duration of an image sensor used in the optical observation system.
nonlinearities of an image sensor used in the optical observation system.
distance of an illumination system from the object regions. This distance may be determined for example based on the information about the depth distribution of the object regions and the information about the orientation of the object regions, and the position and orientation of the illumination system in relation to the observation object. By way of example, a navigation system, stereography, etc. may be used to determine the position and orientation of the illumination system in relation to the observation object.
orientation of an illumination system in relation to the object regions. The orientation, like the distance, may be determined for example based on the information about the depth distribution of the object regions and the information about the orientation of the object regions, and the position and orientation of the illumination system in relation to the observation object.
intensity of an illumination light source.
spectral intensity distribution of an illumination light source.
zoom setting of an illumination zoom.
position of an illumination stop.

In a fourth of the advantageous developments, the evaluation device is designed to generate a graphical display that displays the object regions in which the determined expected fluorescence intensity is sufficient to be able to be detected by the optical observation system with the given sensitivity thereof.

In a fifth of the advantageous developments, the optical observation system comprises an optimization unit that is designed, based on the simulation, to determine an improved parameter value, in particular an optimized parameter value, for the at least one parameter such that the expected fluorescence intensity simulated with the improved or optimized parameter value is sufficient, in as many object regions as possible (where possible in all object regions), to be able to be detected by the optical observation system with the given sensitivity thereof.

In a sixth of the advantageous developments, the optical observation system comprises a control unit for controlling the optical observation system, which control unit is connected to the optimization unit in order to receive the improved or optimized parameter value and is designed to set the at least one parameter to the improved or optimized parameter value.

In a seventh of the advantageous developments, the optical observation system comprises a compensation factor determination unit that is designed to determine a compensation factor by way of which, in a digital image recorded by an image sensor, it is possible to compensate for a change in the fluorescence intensity caused by a deviation of the parameter value of the at least one parameter which influences the observation of the fluorescence intensity from a reference parameter value. The compensation factor determination unit is furthermore designed to determine the compensation factor on the basis of at least one reference value for the fluorescence intensity as determined for a reference concentration of the fluorescent dye and for a reference parameter value and of a simulation of the fluorescence intensity expected for the respective object regions, wherein, in the simulation, a change in the fluorescence intensity in comparison to the reference intensity is determined for a deviation of the parameter value of the at least one parameter which influences the observation of the fluorescence intensity from the reference parameter value.

According to the invention, a computer-implemented method for preparing the observation of a fluorescence intensity of fluorescence radiation of a fluorescent dye in an observation object that comprises object regions that differ from one another in terms of their depth and/or their orientation is also provided, wherein the observation is intended to be implemented using an optical observation system, using which fluorescence radiation is able to be observed, provided that this has a certain minimum intensity. The method comprises the following steps:
receiving or retrieving the parameter value of at least one parameter which influences the observation of the fluorescence intensity, and
simulating the fluorescence intensity expected for the respective object regions on the basis of the determined parameter value of the at least one parameter and a model of the influence of the at least one parameter on the fluorescence intensity.

Within the scope of the simulation, a minimum concentration of the fluorescent dye is predefined and the fluorescence intensity expected with the minimum concentration is determined for each object region based on the simulation.

According to the invention, a computer program for preparing the observation of a fluorescence intensity of fluorescence radiation of a fluorescent dye in an observation object that comprises object regions that differ from one another in terms of their depth and/or their orientation is also provided, wherein the observation is intended to be implemented using an optical observation system, using which fluorescence radiation is able to be observed, provided that this has a certain minimum intensity. The computer program comprises instructions which, when executed on a computer, prompt the latter to
receive or retrieve the parameter value of at least one parameter which influences the observation of the fluorescence intensity, and
simulate the fluorescence intensity expected for the respective object regions on the basis of the received or retrieved parameter value of the at least one parameter and a model of the influence of the at least one parameter on the fluorescence intensity.

Within the scope of the simulation, a minimum concentration of the fluorescent dye is predefined and the fluorescence intensity expected with the minimum concentration is determined for each object region based on the simulation.

Finally, according to the invention, a data processing unit for preparing the observation of a fluorescence intensity of fluorescence radiation of a fluorescent dye in an observation object that comprises object regions that differ from one another in terms of their depth and/or their orientation is provided, wherein the observation is intended to be implemented using an optical observation system, using which fluorescence radiation is able to be observed, provided that this has a certain minimum intensity. The data processing unit comprises a memory and a processor, and the processor, by means of a computer program stored in the memory, is designed to
receive or retrieve the parameter value of at least one parameter which influences the observation of the fluorescence intensity, and
simulate the fluorescence intensity expected for the respective object regions on the basis of the received or retrieved parameter value of the at least one parameter and a model of the influence of the at least one parameter on the fluorescence intensity.

Within the scope of the simulation, a minimum concentration of the fluorescent dye is predefined and the fluorescence intensity expected with the minimum concentration is determined for each object region based on the simulation.

The advantages of the computer-implemented method according to the invention, of the computer program according to the invention, and of the data processing unit according to the invention are evident from the method according to the invention and are therefore not described in more detail here. Moreover, the computer-implemented method according to the invention, the computer program according to the invention, and the data processing unit according to the invention may be developed so that they allow the implementation of the developments of the method according to the invention.

Further features, properties and advantages of the present invention will become apparent from the following description of exemplary embodiments with reference to the accompanying figures.

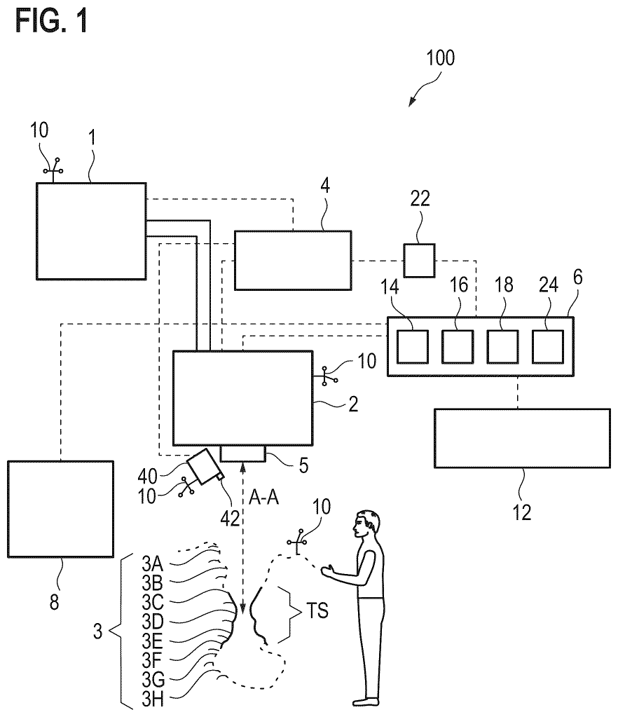

FIG. 1 shows an embodiment of an optical observation system for observing a fluorescence intensity of fluorescence radiation.

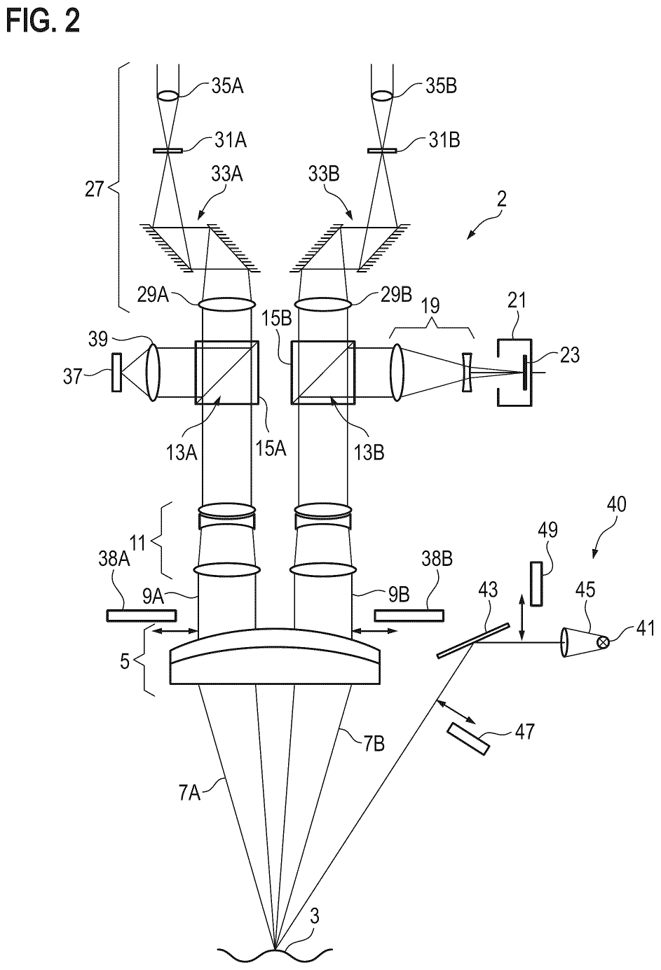

FIG. 2 shows the optical components of a surgical microscope as may find use as an optical observation device in the optical observation system, in a schematic illustration.

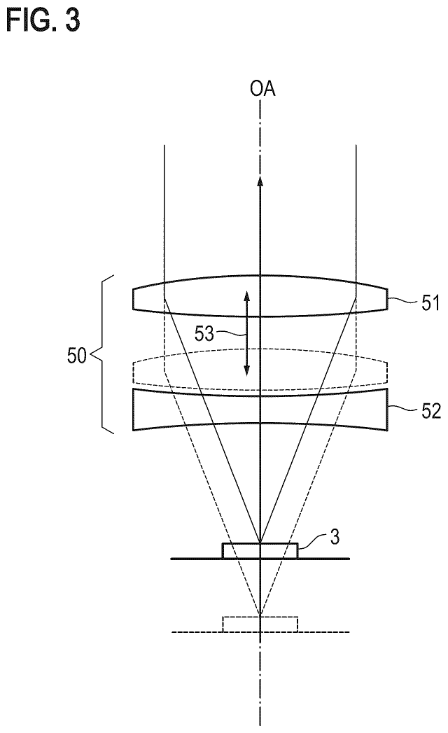

FIG. 3 schematically shows the basic structure of a varioscope objective.

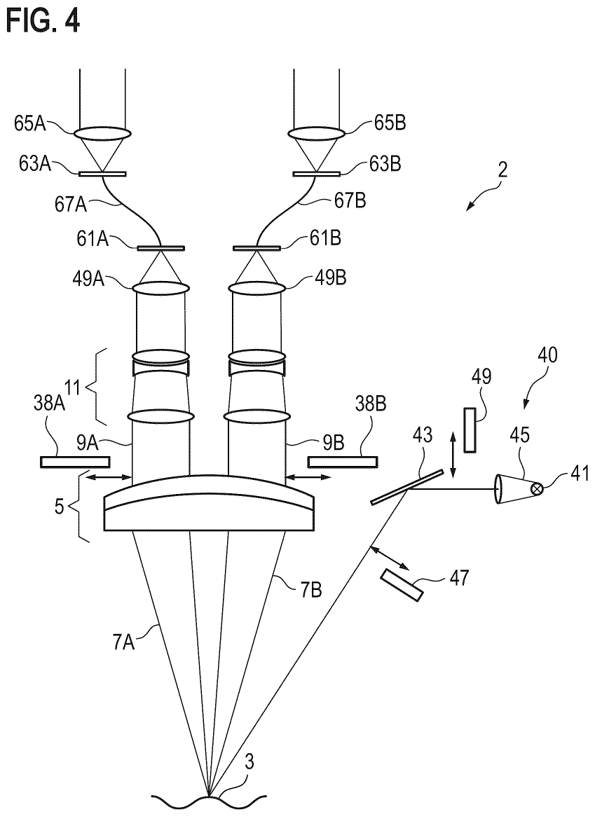

FIG. 4 shows the optical components of a digital surgical microscope as may find use as an optical observation device in the optical observation system, in a schematic illustration.

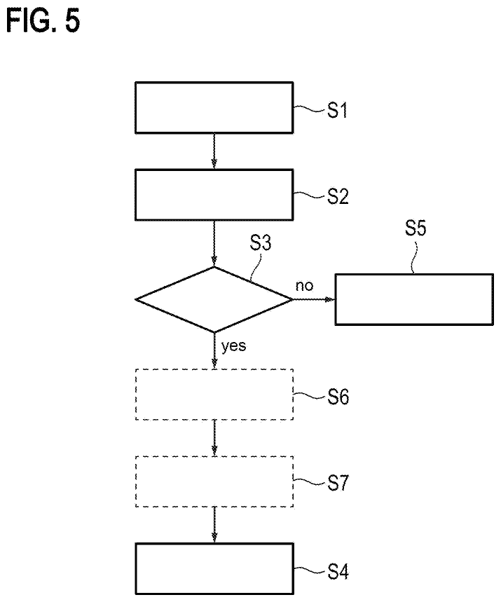

FIG. 5 shows an exemplary embodiment of a method for preparing the observation of a fluorescence intensity.

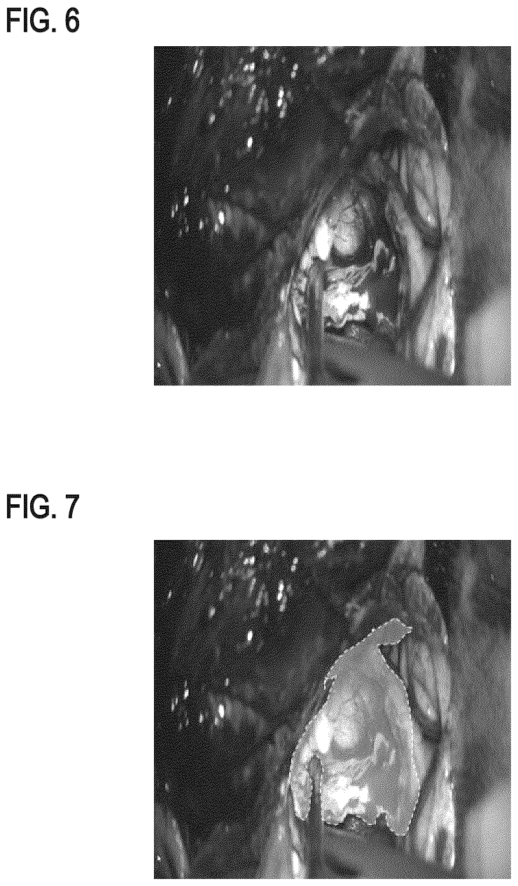

FIG. 6 shows an example of an observation object.

FIG. 7 shows the observation object from FIG. 6 with a marking indicating where the expected fluorescence intensity is not sufficient to be able to be detected by the optical observation device given the sensitivity thereof.

FIG. 1 shows an exemplary embodiment of an optical observation system 100 according to the invention, in a very schematic illustration. In the present exemplary embodiment, the optical observation system 100 comprises a surgical microscope 2 which is mounted on a stand 1, typically a robotic stand, and which serves as an optical observation device used to observe an observation object 3. The observation object 3 has an irregular structure with object regions 3A-H, which have different orientations and are at different depths. As a consequence, the object regions 3A-H have different distances from and orientations in relation to the surgical microscope 2. It should be observed here that the object regions are only depicted in a part of the observation object 3 in FIG. 1 for reasons of clarity.

The optical observation system 100 comprises a controller 4 and a data processing unit 6 that prepares digital image data, recorded using an image sensor present in the surgical microscope 2, for display on a monitor 8, which may be a 3-D monitor in particular, and that outputs said data to the monitor 8. Further display units, for example an HMD (head-mounted display) or a digital eyepiece of the surgical microscope 2, may be present instead of the monitor 8 or in addition to the monitor, the data processing unit 6 transmitting to said further display units the digital image data prepared for display. In the present exemplary embodiment, loading a computer program may configure the data processing unit 6 to carry out a method for preparing the observation of a fluorescence intensity of fluorescence radiation of a fluorescent dye in the observation object 3. Alternatively, the data processing unit 6 may contain an application-specific integrated circuit (ASIC), in which the steps for carrying out the method are stored.

The optical observation system 100 moreover comprises an illumination system 40, by means of which the observation object 3 may be illuminated with illumination light. In the present exemplary embodiment, the illumination system 40 is able to illuminate the observation object 3 using a specific wavelength, referred to hereinafter as excitation wavelength, which excites a fluorescent dye, introduced into the observation object 3, to emit fluorescence radiation. The surgical microscope 2 is configured to detect the fluorescence radiation emanating from the fluorescent dye. To this end, the surgical microscope 2 may comprise in particular a digital image sensor with a sufficient sensitivity to the wavelength of the fluorescence of the fluorescent dye. Moreover, at least one filter that is introducible into the observation beam path is present in the present exemplary embodiment and may be used to remove light at the excitation wavelength, which is reflected by the observation object 3, from the observation beam path. As a rule, the excitation is implemented at an intensity which would lead to the fluorescence radiation being swamped by the light at the excitation wavelength. This swamping may be prevented by the at least one filter that is introducible into the observation beam path.

Naturally, the fluorescence need not necessarily be observed by a display such as for example the monitor 8; instead, it may also be observed purely optically by way of eyepieces of the surgical microscope. Independently of whether the fluorescence is observed by means of eyepieces or with the aid of image sensors, the fluorescence intensity must exceed a certain minimum level in order to be able to be detected. In the case of an image sensor, the detection threshold in the corresponding wavelength range must be exceeded by the fluorescence intensity. However, a certain minimum intensity of the fluorescence radiation is required in the case of the visual observation as well, so that the eye as a detector may perceive the fluorescence radiation. The main field of use of the present invention, however, is the observation of fluorescence with the aid of digital image sensors such as CCD sensors or CMOS sensors.

The optical components of a surgical microscope 2, which may find use as an optical observation device in the optical observation system 100, are explained hereinafter with reference to FIGS. 2 to 4. However, the invention may also be realized with other optical observation devices such as cameras or endoscopes, for example.

As essential optical constituent parts, the surgical microscope 2 shown in FIG. 2 comprises an objective 5 which is intended to face an observation object 3 and which may be embodied as an achromatic or apochromatic objective in particular. In the present embodiment, the objective 5 consists of two partial lenses that are cemented to one another and form an achromatic objective. The observation object 3 is arranged in the focal plane of the objective 5 such that it is imaged at infinity by the objective 5. In other words, a divergent beam 7 emanating from the observation object 3 is converted into a parallel beam 9 during its passage through the objective 5. In this case, the image of the observation object is sufficiently focused in a depth range around the focal plane so as to be perceived in focus with the image sensor or the eye. Thus, this depth range is referred to as depth of focus and is denoted by reference sign “TS” in FIG. 1.

A magnification changer 11 is arranged on the observer side of the objective 5 and may be embodied either as a zoom system for changing the magnification factor in a continuously variable manner as in the illustrated exemplary embodiment, or as what is known as a Galilean changer for changing the magnification factor in a stepwise manner. In a zoom system, constructed by way of example from a lens combination having three lenses, the two object-side lenses may be displaced in order to vary the magnification factor. In actual fact, however, the zoom system also may have more than three lenses, for example four or more lenses, in which case the outer lenses then may also be arranged in a fixed manner. In a Galilean changer, by contrast, there are a plurality of fixed lens combinations which represent different magnification factors and which may be introduced into the beam path in alternation. Both a zoom system and a Galilean changer convert an object-side parallel beam into an observer-side parallel beam with a different beam diameter. In the present embodiment, the magnification changer 11 already is part of the binocular beam path of the surgical microscope 2, which is to say it has a dedicated lens combination for each stereoscopic partial beam path 9A, 9B of the surgical microscope 2. In the present embodiment, a magnification factor is adjusted by means of the magnification changer 11 by way of a motor-driven actuator (not depicted here) which, together with the magnification changer 11, is part of a magnification changing unit for adjusting the magnification factor.

In the present exemplary embodiment, the magnification changer 11 is adjoined on the observer side by an interface arrangement 13A, 13B, by means of which external devices may be connected to the surgical microscope 1 and which comprises beam splitter prisms 15A, 15B in the present exemplary embodiment. However, in principle, use may also be made of other types of beam splitters, for example partly transmissive mirrors. In the present exemplary embodiment, the interfaces 13A, 13B serve to output couple a beam from the beam path of the surgical microscope 2 (beam splitter prism 15B) and to input couple a beam into the beam path of the surgical microscope 2 (beam splitter prism 15A). However, they may both also be embodied to output couple a beam from the beam path of the surgical microscope 2 or both be embodied to input couple a beam into the beam path of the surgical microscope 2.

In the present exemplary embodiment, the beam splitter prism 15A in the partial beam path 9A serves to mirror information or data for an observer via the beam splitter prism 15A into the partial beam path 9A of the surgical microscope 1 with the aid of a display 37, for example a digital mirror device (DMD) or an LCD display, and an associated optical unit 39. A camera adapter 19 with a camera 21 fastened thereto, said camera being equipped with an electronic image sensor 23, for example with a CCD sensor or a CMOS sensor, is arranged at the interface 13B in the other partial beam path 9B. It is possible by means of the camera 21 to record an electronic image and, in particular, a digital image of the observation object 3. The image sensor used may also be, in particular, a hyperspectral sensor comprising not just three spectral channels (e.g., red, green and blue), but rather a multiplicity of spectral channels. If both interfaces 13A, 13B are embodied to output couple a beam from the beam path of the surgical microscope 2, a camera adapter 19 with a camera 21 fastened thereto may be arranged on both interfaces 13A, 13B in each case. This allows spectroscopic images to be recorded.

Moreover, at least one further interface arrangement with at least two beam splitters may be present in the surgical microscope, wherein for example the one interface arrangement may serve to input couple stereoscopic partial images and the other interface arrangement may serve to output couple stereoscopic partial images.

The interface 13 is followed on the observer side by a binocular tube 27. The latter has two tube objectives 29A, 29B, which focus the respective parallel beam 9A, 9B onto an intermediate image plane 31, which is to say image the observation object 3 onto the respective intermediate image plane 31A, 31B. Finally, the intermediate images situated in the intermediate image planes 31A, 31B are imaged in turn at infinity by eyepiece lenses 35A, 35B, with the result that an observer may observe the intermediate image with a relaxed eye. Moreover, the distance between the two partial beams 9A, 9B is increased in the binocular tube by means of a mirror system or by means of prisms 33A, 33B in order to adapt said distance to the interocular distance of the observer. In addition, image erection is carried out by the mirror system or the prisms 33A, 33B.

The surgical microscope 2 moreover is equipped with an illumination system 40, by means of which the observation object 3 may be illuminated with illumination light. To this end, the illumination system 40 comprises a white-light source 41, for instance a halogen lamp or a gas discharge lamp such as for example a xenon lamp, in the present example. However, LEDs may also be considered for the light sources. The light emanating from the white-light source 41 is steered in the direction of the observation object 3 via a deflection mirror 43 or a deflection prism in order to illuminate said observation object. Furthermore, an illumination optical unit 45 is present in the illumination system 40 and ensures a uniform illumination of the entire observed observation object 3. Here, the illumination optical unit 45 may also comprise a zoom system (illumination zoom), which is able to modify the size of the illumination light spot, and/or a system which allows the focal distance of the illumination optical unit 45 to be varied. Moreover, the illumination system 40 may be equipped with a light source for emitting light at a wavelength which excites a fluorescence in a fluorescent dye that has been introduced into the observation object 3. Alternatively, as depicted in FIG. 2, a spectral filter 47 may be present; the latter may be introduced into the illumination beam path and substantially only allows passage of the wavelength of the light from the white-light source 41 which excites the fluorescence in the fluorescent dye. Moreover, filters 38A, 38B which block the wavelength exciting the fluorescence in the fluorescent dye are introduced into the observation beam path.

The illumination system 40 may moreover comprise at least one stop and/or at least one lens, by means of which it is possible to influence the profile of the illumination light cone emanating from the light source, for example in order to generate an illumination profile with emphasis on the center. The at least one stop and/or the at least one lens 49 may be introduced into the illumination beam path when necessary. Furthermore, the illumination system 40 may comprise stops that may bring about a sharp delimitation of the luminous field in the observation object 3.

Reference is made to the fact that the illumination beam path depicted in FIG. 2 is highly schematic and does not necessarily reproduce the actual course of the illumination beam path. In principle, the illumination beam path may be embodied as what is known as oblique illumination, which comes closest to the schematic illustration in FIG. 2. In the case of such oblique illumination, the beam path extends at a relatively large angle (6° or more) with respect to the optical axis of the main objective 5 and, as depicted in FIG. 2, may extend completely outside the main objective 5. Alternatively, however, there is also the possibility of allowing the illumination beam path of the oblique illumination to extend through a marginal region of the main objective 5. A further possibility for the arrangement of the illumination beam path is what is known as 0° illumination, in which the illumination beam path extends through the main objective 5 and is input coupled into the main objective 5 between the two partial beam paths 9A, 9B, along the optical axis of the main objective 5, in the direction of the observation object 3. Finally, it is also possible to design the illumination beam path as what is known as coaxial illumination, in which a first illumination partial beam path and a second illumination partial beam path are present. The illumination partial beam paths are input coupled into the surgical microscope 2 in a manner parallel to the optical axes of the observation partial beam paths 9A, 9B by way of one or more beam splitters, with the result that the illumination extends coaxially in relation to the two observation partial beam paths.

In the embodiment variant of the surgical microscope 2 shown in FIG. 2, the objective 5 only consists of an achromatic lens with a fixed focal length. However, use may also be made of an objective lens system made of a plurality of lenses, in particular a so-called varioscope objective, by means of which it is possible to vary the working distance of the surgical microscope 2, which is to say the distance between the object-side focal plane and the vertex of the first object-side lens surface of the objective 5, also referred to as front focal distance. The observation object 3 arranged in the focal plane is imaged at infinity by the varioscope objective 50, too, and so a parallel beam is present on the observer side.

One example of a varioscope objective is depicted schematically in FIG. 3. The varioscope objective 50 comprises a positive member 51, which is to say an optical element with positive refractive power, depicted schematically as a convex lens in FIG. 3. Moreover, the varioscope objective 50 comprises a negative member 52, which is to say an optical element with negative refractive power, depicted schematically as a concave lens in FIG. 3. The negative member 52 is situated between the positive member 51 and the observation object 3. In the illustrated varioscope objective 50, the negative member 52 has a fixed arrangement, whereas, as indicated by the double-headed arrow 53, the positive member 51 is arranged to be displaceable along the optical axis OA. When the positive member 51 is displaced into the position illustrated by dashed lines in FIG. 3, the back focal length increases, and so there is a change in the working distance of the surgical microscope 2 from the observation object 3.

Even though the positive member 51 has a displaceable configuration in FIG. 3, it is also possible, in principle, to arrange the negative member 52 to be displaceable along the optical axis OA instead of the positive member 51. However, the negative member 52 often forms the last lens of the varioscope objective 50. A stationary negative member 52 therefore offers the advantage of making it easier to seal the interior of the surgical microscope 2 from external influences. Furthermore, it is noted that even though the positive member 51 and the negative member 52 in FIG. 3 are only illustrated as individual lenses, each of these members may also be realized in the form of a lens group or a cemented element instead of in the form of an individual lens, for example to embody the varioscope objective 50 to be achromatic or apochromatic.

FIG. 4 shows a schematic illustration of an example of a purely digital surgical microscope 48. In this surgical microscope 48, the main objective 5, the magnification changer 11, and the illumination system 40 do not differ from the surgical microscope 2 depicted in FIG. 2. The difference lies in the fact that the surgical microscope 48 shown in FIG. 4 does not comprise an optical binocular tube. Instead of the tube objectives 29A, 29B from FIG. 2, the surgical microscope 48 depicted in FIG. 4 comprises focusing lenses 49A, 49B which image the binocular observation beam paths 9A, 9B onto digital image sensors 61A, 61B. Here, the digital image sensors 61A, 61B may be CCD sensors or CMOS sensors, for example. The images recorded by the image sensors 61A, 61B are digitally transmitted to a data processing unit 6, as depicted in FIG. 1, which prepares said images for display on a monitor 8 or on digital displays 63A, 63B and then transmits said prepared images to the monitor 8 or the digital displays 63A, 63B. The digital displays 63A, 63B may be designed as LED displays, as LCD displays or as displays based on organic light-emitting diodes (OLEDs). Like in the present example, they may be assigned to eyepiece lenses 65A, 65B, by means of which the images displayed on the displays 63A, 63B are imaged at infinity such that an observer is able to observe said images with relaxed eyes. The displays 63A, 63B and the eyepiece lenses 65A, 65B may be part of a digital binocular tube; however, they may also be part of a head-mounted display (HMD) such as for instance a pair of smartglasses. In particular, the monitors or displays 63A, 63B may be designed for the observation of stereoscopic images. To this end, the displays 63A, 63B may be assigned to different eyes of the user and represent stereoscopic partial images. In the case of the monitor 8, the stereoscopic partial images may be depicted sequentially in time. By means of synchronized shutter glasses, for example, the stereoscopic partial images may then be displayed, exclusively in each case, to the appropriate eye. An alternative consists of depicting the stereoscopic partial images in differently polarized light and equipping the observer with a pair of glasses which, for the right and the left eye, in each case only allows polarized light from one of the stereoscopic partial images to pass.

Even though FIG. 4, like FIG. 2, depicts only one achromatic lens 5 with a fixed focal length, the surgical microscope 48 shown in FIG. 4 may comprise a varioscope objective instead of the objective lens 5, like the surgical microscope 2 illustrated in FIG. 2. Furthermore, FIG. 4 shows a transmission of the images recorded by the image sensors 61A, 61B to the displays 63A, 63B by means of cables 67A, 67B. Instead of in wired fashion, the images may however also be transmitted wirelessly to the displays 63A, 63B, especially if the displays 63A, 63B are part of a head-mounted display.

In the exemplary embodiment shown in FIG. 1, the robotic stand 1, the surgical microscope 2, and the illumination system 40 each have a tracking target 10, with the aid of which a tracking system 12 is able to determine the position and orientation of the respective component. Moreover, a tracking target 12 is also attached directly or indirectly to the observation object 3, with the result that the position and orientation of the respective component may be determined in relation to the observation object 3. By way of example, the tracking target 12 may be fixed to a skull clamp for an indirect connection to the observation object 3.

A first exemplary embodiment of a method for preparing the observation of a fluorescence intensity using the optical observation system 100 depicted in FIG. 1 is described hereinafter with reference to FIG. 1 and FIG. 5, the latter showing a flowchart of the method.

The observation of fluorescence is important within the scope of tumor resection in particular, since the treating surgeon uses the fluorescence to assess which tissue is tumor tissue and therefore needs to be removed. However, this assumes that the fluorescence intensity of the tumor tissue is sufficient so as to be able to be detected throughout tumor tissue. However, some types of tumors, for example low-grade glioma (LLG), accumulate only very small amounts of contrast agent (e.g., PPIX in the case of low-grade glioma), with the result that the fluorescence intensity is very low and correspondingly difficult to measure. Even in the case of sensitive optical observation devices, the fluorescence intensity of PPIX in low-grade gliomas for example is often close to the detection threshold, and so optimal observation conditions must be ensured in order to be able to detect a reliable fluorescence signal using the image sensor 23 or the image sensors 61A, 61B. Thus, the prior art has seen cases where tissue regions located closer to the surgical microscope 2 (e.g., object regions 3A-C) appear to fluorescent since sufficient excitation light arrives at the tissue and enough of the emitted fluorescence is guided to the image sensor 23 or the image sensors 61A, 61B. However, tissue regions lower down (e.g., object regions 3D-H) in the same scene might no longer be illuminated sufficiently brightly with the excitation wavelength, despite having the same concentration of fluorescent dye, on account of the greater distance of the illumination light source 41, and/or the collection efficiency decreases on account of the greater distance of the surgical microscope 2 from the corresponding object regions 3D-H, for example with a (1/distance)∝dependence overall. If the fluorescence intensity of the higher-up object regions 3A-C is just above the sensitivity threshold of the image sensor 23 or image sensors 61A, 61B, then the same fluorescence intensity at tissue regions 3D-H lower down might just no longer be able to be detected by the image sensor 23 or image sensors 61A, 61B. That is to say, the situation may arise in which the image obtained by the image sensor 23 or image sensors 61A, 61B may contain some object regions 3A-C of the observation object 3 depicted in the image which are able to be illuminated and observed more efficiently and hence are depicted as a fluorescent, while other object regions 3D-H with the same concentration of fluorescent dye are depicted as non-fluorescent. This may lead to significant challenges for the treating surgeon, and these may unnecessarily lengthen the treatment. Additionally, the fluorescence may suddenly disappear or appear in the image obtained by the image sensor 23 or image sensors 61A, 61B following an adjustment to the surgical microscope 2 (e.g., change in zoom level, movement of the surgical microscope and/or of the illumination system 40 relative to the observation object 3), making a reliable diagnosis significantly more difficult.

The data processing unit 6 of the optical observation system 100 from FIG. 1 therefore comprises a determination device 14 for determining the parameter value of at least one parameter which influences the observation of the fluorescence intensity. Moreover, it comprises a simulation device 16 for simulating the fluorescence intensity expected for the respective object regions 3A-H on the basis of the determined values of a model of the influence of the at least one parameter on the fluorescence intensity. For each object region 3A-H, an evaluation device 18 of the data processing unit 6 then determines the expected fluorescence intensity for a given minimum concentration of the fluorescent dye. In the process, there may also be check as to whether the determined fluorescence intensity is sufficient for detection by the image sensor 23 or image sensors 61A, 61A given the sensitivity thereof. If the evaluation device 18 determines that there are object regions 3A-H for which the minimum concentration of fluorescent dye does not lead to a signal that is detectable by the image sensor 23 or image sensors 61A, 61B, then the evaluation device 18 in the present exemplary embodiment outputs an alert which communicates to the user that a reliable detection of the fluorescence for the minimum concentration is not ensured.

In an optional development, the evaluation device 18 may generate a graphical display, from which it is possible to read the object regions 3A-H in which the expected fluorescence intensity is not sufficient to be able to be detected by the image sensor 23 or image sensors 61A, 61B. In the simplest case, such a graphical display may be a contour line 20 which is superimposed on an image of the observation object 3, as shown in FIG. 6, and which surrounds those object regions in which the fluorescence intensity is not sufficient to be able to be detected by the image sensor 23 or images sensors 61A, 61B (cf. FIG. 7). An alternative graphical display may be implemented in the form of a map. By way of example, object regions in which the minimum concentration of fluorescent dye leads to a detectable fluorescence intensity at the image sensor 23 or image sensors 61A, 61B may be colored differently in such a map than object regions for which the minimum concentration of fluorescent dye does not lead to a detectable fluorescence intensity at the image sensor 23 or image sensors 61A, 61B. Optionally, the map may have a color transition which specifies how “far away” the respective object regions still are from a reliable detection of the fluorescence intensity by the image sensor 23 or image sensors 61A, 61B. By way of example, object regions whose fluorescence intensity is only 10% below the fluorescence intensity required for detection by the image sensor 23 or image sensors 61A, 61B might be depicted in green while object regions at 50% below the required value of fluorescence intensity are colored red. The user may make a decision on the basis of the graphical display as to whether the object regions in which the fluorescence intensity is not sufficient to be able to be detected by the image sensor 23 or image sensors 61A, 61B are relevant to the sought-after observation purpose, for instance a tumor resection. If the observed region of the observation object 3 is so large that it comprises object regions known, for example from preliminary examinations, not to contain tumor tissue, then it may be acceptable in these regions for the minimum concentration of fluorescent dye not to lead to a fluorescence intensity detectable by the image sensor 23 or image sensors 61A, 61B.

Moreover, there is the option of the evaluation device 18 determining an improved parameter value for at least one parameter of the surgical microscope 2 and/or illumination system 40 and/or image sensor 23 or image sensors 61A, 61B by resorting to the simulation device 16, said improved parameter value leading to an increase in the number of object regions 3A-H in which the fluorescence intensity of the minimum concentration of fluorescent dye leads to a detectable signal at the image sensor 23 or image sensors 61A, 61B. The at least one improved parameter value may then either be set in automated fashion or be presented to the user as a settings recommendation, for example on the monitor 8.

In the present exemplary embodiment, the determination device 14, the simulation device 16, and the evaluation device 18 are integrated in the data processing unit 6 as software modules. However, they may also be integrated in the controller 4 of the optical observation system 100 or in a PC associated with the optical observation system 100. Further, there is the option of the determination device 14, the simulation device 16, and the evaluation device 18 to be integrated in different components of the optical observation system 100. By way of example, the determination device 14 may be integrated in the controller 4 and the simulation device 16 and the evaluation device 18 may be integrated in a PC. In principle, there is also the option of designing the determination device 14 and/or the simulation device 16 and/or the evaluation device 18 as an independent hardware module.

In the present exemplary embodiment, the determination device 14, the simulation device 16, and the evaluation device 18 essentially carry out three steps. These are the acquisition of parameter values for parameters which influence the fluorescence intensity arriving at the image sensor (determination device 14 in step S1), the simulation of the fluorescence intensity arriving at the image sensor 23 or image sensors 61A, 61B from the respective object region 3A-H, with use being made of a model of the fluorescence observation which depends on the parameters for which the parameter values are acquired (simulation device 16 in step S2) in order to determine, for the individual object regions 3A-H, the expected fluorescence intensity at the location of the image sensor 23 or image sensors 61A, 61B for a given minimum concentration of the fluorescent dye, and the evaluation of the simulation (by the evaluation device 18 in step S3) in order to determine the object regions 3A-H at which the expected fluorescence intensity is sufficient to be able to be detected at the location of the image sensor 23 or image sensors 61A, 61B by the image sensor 23 or image sensors 61A, 61B for the given minimum concentration of the fluorescent dye. If the evaluation yields that there are object regions 3A-H present for which the minimum concentration of the fluorescent dye does not lead to a fluorescence intensity detectable by the image sensor 23 or image sensors 61A, 61B at the location thereof, then the evaluation device 18 may output an alert. Moreover, the evaluation device 18 may carry out further tasks, for example the creation of the above-described graphical display or the above-described determination of at least one improved parameter value. The current parameter values of the parameters used in the simulation in step S2 are acquired in step S1. Examples of parameters which in the present exemplary embodiment may be included in the simulation include the distance of the surgical microscope 2 from the object regions 3A-H of the observation object 3, the orientation of the surgical microscope 2 in relation to the object regions 3A-H of the observation object 3, the zoom setting of the surgical microscope 2, the front focal distance of the surgical microscope 2, the stop setting of the surgical microscope 2, the gain of the image sensor 23 used in the surgical microscope 2 or of the image sensors 61A, 61B used in the surgical microscope, the exposure duration of the image sensor 23 used in the surgical microscope 2 or of the image sensors 61A, 61B used in the surgical microscope, nonlinearities of the image sensor 23 used in the surgical microscope 2 or of the image sensors 61A, 61B used in the surgical microscope, the distance of the illumination system 40 from the object regions 3A-H of the observation object 3, the orientation of the illumination system 40 in relation to the object regions 3A-H of the observation object 3, the intensity of an illumination light source 41, the spectral intensity distribution of an illumination light source 41, the zoom setting of an illumination zoom, and the position of an illumination stop.

To acquire the parameter values, the data processing unit 6 of the present exemplary embodiment comprises a parameter value determination device 14, which retrieves the parameter values set at the surgical microscope 2, at the stand 1, and at the illumination system 40 from the controller 4. However, the parameter values may alternatively also be acquired by reading sensor measurement values. There is also the option of calculating parameter values indirectly from other parameter values that are acquired directly. By way of example, the relative position and the relative orientation of the surgical microscope 2 in relation to the observation object 3 may be calculated from the position and orientation of the surgical microscope 2, acquired in the coordinate system of the tracking system 12 by means of the tracking system 12, and the position and orientation of the observation object, likewise acquired in the coordinate system of the tracking system 12 by means of the tracking system 12. Alternatively, the positions and the orientation of the surgical microscope 2 in relation to the object regions 3A-H of the observation object 3 may be determined by means of stereographic methods from stereoscopic images of the observation object. Variables such as the zoom setting of the surgical microscope 2 or illumination system 40 may be read by the determination unit 14 directly from the controller 4 of the surgical microscope 2 and illumination system, like in the present exemplary embodiment, or may be acquired by sensors in the respective zoom system. The intensity and the spectral intensity distribution of the illumination light source 41 may also be acquired indirectly by virtue of being calculated on the basis of characteristics from the values of the service life counter, which registers the service life of the illumination light source to date.

On account of a degradation of the illumination light source 41 over time, the intensity of the excitation wavelength currently output by the illumination light source 41 deviates from the nominally set intensity of the excitation wavelength over time. The nominally set intensity of the excitation wavelength in this case emerges from the nominally set intensity of the illumination light source 41 and the spectral intensity distribution thereof. In the present exemplary embodiment, the intensity of the excitation wavelength currently output by the illumination light source 41 may be determined by means of an intensity sensor 42 which is installed in the surgical microscope 2 or in the illumination system 40. The intensity sensor 42 is preferably sensitive exclusively, or at least predominantly, to the excitation wavelength in order to obtain the most accurate acquisition possible of the intensity of the excitation wavelength for the fluorescence. Optionally, the spectral intensity distribution of the illumination light may also be determined by means of an intensity sensor, for instance with the aid of a multispectral sensor. The intensity currently output by the illumination light source 41 at the excitation wavelength (or the spectral intensity distribution of the illumination light) given the nominally set intensity of said illumination light source may however alternatively or additionally also be determined by virtue of a calibration target (e.g., a white sheet of paper), placed under the surgical microscope 2 and focused prior to the actual observation, being recorded by the image sensor 23 or image sensors 61A, 61B and the signal generated in the image sensor 23 or image sensors 61A, 61B in the process being evaluated. Preferably, only the color channel/channels of the image sensor or sensors that comes/come closest to the excitation wavelength is/are evaluated. The determined intensity may then be used in the simulation in step S2. However, the deviation of the current intensity of the excitation wavelength from the intensity of the excitation wavelength arising from the nominally set intensity of the illumination light source 41 may optionally also be determined from the determined intensity of the excitation wavelength and a correction value may be determined, with the setting of the illumination light source 41 having to be corrected by said correction value in order to obtain a current intensity of the excitation wavelength which corresponds to the nominal intensity of the excitation wavelength. Thus, the intensity of the illumination light source 41 may be adapted in such a way by means of the correction value that the degradation of the illumination light source 41 is just compensated for within the scope of the fluorescence excitation.

A further alternative or additional option for determining the intensity of the excitation wavelength currently given off by the illumination light source 41 having the nominally set intensity lies in the determination thereof via a service life counter of the illumination light source 41 (typically a xenon lamp or LED) and the normally set intensity for the illumination light source 41. As the service life of the illumination light source increases, the intensity of the illumination light emitted thereby decreases, with the result that its actual intensity deviates from the nominally set intensity. The current intensity of the illumination light source 41 may be determined from the nominally set intensity on the basis of the service life of the illumination source 41 to date and may be used in the simulation in step S2. Optionally, it is possible here, too, to determine a correction value from the degree by which the current intensity of the illumination light source 41 has been reduced vis-à-vis the nominally set intensity, said correction value specifying the amount by which the intensity of the illumination light source 41 must be corrected in order to obtain a current intensity that corresponds to the nominally set intensity. Thus, the intensity of the illumination light source 41 may be adapted in such a way by means of the correction value that the degradation of the illumination light source 41 is just compensated for within the scope of the fluorescence excitation. In addition to the change in the overall intensity of the illumination source 41, the utilized degradation model of the illumination light source 41 may optionally also take account of the shifts in the spectral intensity distribution of the illumination radiation that occur over time. The shift in the spectral intensity distribution leads to the degree by which the current intensity of the illumination light source 41 is reduced vis-à-vis the nominally set intensity being dependent on the wavelength. The current intensity may be determined for each wavelength of the illumination light by taking account of the shifts in the spectral intensity distribution that occur over time. By taking account of the shifts in the spectral intensity distribution that occur over time, it is moreover possible to determine a correction value matched precisely to the excitation wavelength of the fluorescence. On the basis of the corrected excitation wavelength, it is possible to perform a calculation as to how much fluorescence radiation is emitted. In the calculation process, the spectral intensity distribution of the illumination light source 41 may be weighted by the effective spectral excitation curve of the fluorescence. On the basis of such weighting, it is possible within the scope of the calculation of the at least one correction value to take account of the wavelength-dependent properties of the observation object 3, for example the absorption of the excitation wavelength upon penetration into the observation object.

The optional correction of the setting of the illumination light source 41 may be implemented in automated fashion, with the result that the actual set intensity of the illumination light source always corresponds to the nominally set intensity. However, there is also the option of communicating to the user the manner in which the setting of the illumination light source 41 needs to be adapted in order to obtain a desired intensity of the illumination light source.

In step S2, there then is a simulation of the fluorescence emission on the basis of a model of the observation of the fluorescence intensity, with a concentration of fluorescent dye corresponding to a given minimum concentration being assumed for the simulation. The model includes parameters of the illumination system 40, parameters of the surgical microscope 2, and parameters of the image sensor 23 or image sensors 61A, 61B, which parameters are considered in detail hereinafter. In the present exemplary embodiment, the simulation is carried out by a simulation device 16 that is integrated in the data processing unit 6.

The parameters of the illumination system 40 determine, inter alia, the emission intensity of the illumination light source 41 and the emission wavelength of the illumination light source 41, and hence the excitation wavelength. Moreover, the distance of the illumination system 40 from the object regions 3A-H of the observation object 3 and the orientation of the illumination system 40 in relation to the object regions 3A-H of the observation object 3, the zoom setting of the illumination system 40, and the position of stops in the illumination system 40 determine the amount of excitation radiation reaching an object region 3A-H per unit area. This influences the intensity of the excitation wavelength at the location of the fluorescence excitation and hence also the intensity of the fluorescence emission, whereby in turn the fluorescence intensity arriving at the pixels of the image sensor 23 or image sensors 61A, 61B is influenced.

The parameters of the surgical microscope 2 determine how much fluorescence radiation emitted by an object region 3A-H of the observation object 3 reaches the image sensor 23 or image sensors 61A, 61B (or the eyepiece). In this case, the distance of the surgical microscope 2 from the object regions 3A-H of the observation object 3 and the orientation of the surgical microscope 2 in relation to the object regions 3A-H of the observation object 3, the zoom setting in the surgical microscope 2, and the transmission behavior of stops (vignetting) and filters (transmittance) introduced into the observation beam path are of particular importance to the fluorescence intensity that reaches the image sensor 23 or image sensors 61A, 61B, and hence to the signal strength caused in the pixels of the image sensor 23 or image sensors 61A, 61B.

In an image sensor, the gain (amplification factor), the exposure duration, nonlinearities of the image sensor, and other variable factors of the image sensor influence the signal strength caused in the pixels of the image sensor by the incident fluorescence intensity.

Within the scope of the simulation in step S2, an optics and system model is preferably used to take account of the influence of all the aforementioned parameters when calculating the signal strength caused in the pixels of the image sensor 23 or image sensors 61A, 61B when the minimum concentration of fluorescent dye is present.

An example of a possible optics and system model is as follows:

Sensor signal=(1/illumination term)*(1/collection term)*(1/sensor term)*raw signal

In this case, the illumination term arises from the parameter values of the illumination system 40, the collection term arises from the parameter values of the surgical microscope 2, and the sensor term arises from the parameter values of the image sensor 23 or image sensors 61A, 61B.

A possible illumination term is as follows:
Illumination term=[(zoom factor illumination)\hat{ }2*(linear stop-down factor illuminance)/(illumination to object surface distance)\hat{ }2]*luminous_intensity factor_with_spectral_excitation_weight*cos(angle between illumination and surface of the observation object)

A possible collection term is as follows:
Collection term=collection efficiency (zoom factor, focusing)/(objective to surface of the observation object distance)*directional_characteristic_function_of_the_fluorescence_emission (angle of observation with respect to the surface of the observation object)*image location dependent vignetting factor

A possible sensor term is as follows:
Camera term=camera_response_function (exposure time, gain, measured raw signal)

Preferably, shading effects by the observation object 3 are also included when calculating the illumination term and the collection term. For example, this may be implemented with the aid of a stereographically obtained 3-D depth map. Rather than by way of stereography, the 3-D depth map may also be generated using any other method known per se, for example using a depth sensor, by means of structured illumination, etc.

The illumination term, the collection term, and the raw signal are calculated for a respective pixel of the image sensor 23 or image sensors 61A, 61B and the object region 3A-H of the observation object 3 imaged thereon. Thus, in the present exemplary embodiment, the size of an object region 3A-H of the observation object 3 arises from the imaging factor used to image the observation object 3 onto the image sensor 23 or image sensors 61A, 61B, the imaging factor depending inter alia on parameters of the surgical microscope 2, for example on the zoom setting, the working distance, etc. Moreover, the size of the object regions 3A-H which are imaged on a pixel of the image sensor 23 or image sensors 61A, 61B can be increased by virtue of combining a plurality of adjacently arranged pixels to form a larger pixel (known as binning), in order to increase the effective pixel area.

Subsequently, in step S3, the evaluation device 18 evaluates the result of the simulation from step S2 to the effect of whether there are object regions 3A-H present, for which the fluorescence intensity, as determined for the given minimum concentration of the fluorescent dye, is not sufficient for detection by the image sensor 23 or image sensors 61A, 61B at the location of the image sensor 23 or image sensors 61A, 61B. If the evaluation yields that there are object regions 3A-H present for which the minimum concentration of the fluorescent dye does not lead at the location of the image sensor 23 or image sensors 61A, 61B to a fluorescence intensity detectable thereby, then the evaluation device 18 outputs an alert in the present exemplary embodiment (step S4). The alert may optionally be accompanied by a graphical display which indicates the object regions which at the location of the image sensor 23 or image sensors 61A, 61B do not lead to fluorescence intensity detectable by the image sensor 23 or image sensors 61A, 61B, so that the user may estimate the relevance of the alert. If the evaluation yields that there are no object regions which at the location of the image sensor 23 or image sensors 61A, 61B do not lead to a fluorescence intensity detectable by the image sensor 23 or image sensors 61A, 61B, then the preparation of the fluorescence observation is complete and the fluorescence observation may be performed (step S5).

Optionally, the evaluation device 18 may moreover carry out further tasks. By way of example, it may carry out the aforementioned determination of at least one improved parameter value. The optional determination of the at least one improved parameter value is implemented in step S6 either in automated fashion or following a request by the user should the evaluation in step S3 yield that there are object regions 3A-H present which, for the minimum concentration of the fluorescent dye, do not lead at the location of the image sensor 23 or image sensors 61A, 61B to a fluorescence intensity that is detectable by said sensor/sensors. Then, in step S6, the evaluation device 18 determines an improved parameter value for at least one parameter which influences the fluorescence intensity at the location of the image sensor 23 or image sensors 61A, 61B, said parameter value leading to a reduction in the number of object regions 3A-H for which the minimum concentration of the fluorescent dye does not lead to a fluorescence intensity that is detectable by the image sensor 23 or image sensors 61A, 61B at the location of said image sensor or image sensors. To improve the parameter value, the evaluation device 18 may for example vary the corresponding parameter value and calculate a quality value for each value of the parameter occurring during the variation. By way of example, the number of object regions for which the minimum concentration of the fluorescent dye does not lead to a detectable fluorescence intensity at the location of the image sensor 23 or image sensors 61A, 61B may be used as a quality value in this context. Optionally, there may also be weighting in the process as regards to where in the image the remaining object regions for which the minimum concentration of the fluorescent dye does not lead to a detectable fluorescence intensity at the location of the image sensor 23 or image sensors 61A, 61B are located. By way of example, object regions in the center of the image may experience a higher weight than object regions at the edge of the image. Then, the at least one parameter may be improved until the quality value reaches a minimum or drops below a specified value. Alternatively, the number of regions for which the minimum concentration of the fluorescent dye leads to a detectable fluorescence intensity at the location of the image sensor 23 or image sensors 61A, 61B may be used as a quality value. Then, the at least one parameter may be improved until the quality value reaches a maximum or drops below a minimum value. In this case, too, there may be a different weight for object regions in the center of the image vis-à-vis object regions at the edge of the image field. The improved parameter value of the at least one parameter then may either be set in automated fashion (step S7) or be displayed for the user is a recommended setting. By way of example, one of the following improvements may be implemented:
alignment of the viewing angle of the surgical microscope 2 and/or illumination system 40 via the stand 1,
optimization of the luminous intensity of the optical unit of the surgical microscope 2 (zoom setting, working distance),
optimization of the sensitivity of the image sensor 23 or image sensors 61A, 61B,
increase in the illumination brightness.

Within the scope of the method, the user may also be offered the option of setting, for example in the controller 4 or evaluation device 18, which parameters which influence the observation of the fluorescence intensity should be improved automatically by the evaluation device 18 and which they would like to improve manually themselves.

If the concentration of fluorescent dye in the vicinity of the surface should be determined in the fluorescence observation that is carried out following the preparation of the fluorescence observation, then the signal detected by the image sensor 23 or image sensors 61A, 61B must be corrected on the basis of a pre-factor.

Corrected signal=pre-factor*(1/illumination term)*(1/collection term)*(1/sensor term)*raw signal

To this end, the pre-factor introduced into the optics and system model may be determined for each fluorescent dye and for an assumed tissue type by calibration. Preferably, a different pre-factor is used depending on the tissue type. If it is only the fluorescence intensity expected with the minimum concentration that should be determined for each object region, then it is possible to determine a suitable pre-factor on the basis of a model of the emission procedure and the properties of the employed optical system.

Reference objects are used for the calibration. For example, a first reference object is a diffusely reflecting white object, which diffusely reflects 50% of the radiated-in light intensity. By way of example, a second reference object is an aqueous solution in a reference vessel, containing a reference concentration of 100 nM of the fluorescent dye. Moreover, reference poses and orientations of the surgical microscope 2 and the illumination system 40 are defined in relation to the respective reference object. A respective reference parameter value is defined for all parameters of the surgical microscope 2, of the illumination system 40, and of the image sensor 23 or image sensors 61A, 61B that are included in the simulation.

By way of example, the reference parameter values may (but need not) be chosen such that the following reference conditions are present during the calibration:
a perpendicular observation of the surface of the reference object (reference observation angle 90°),
a reference location within the image on the image sensor: The object is located in the center of the image and is imaged onto the central pixel,
a reference working distance of 200 mm between the surgical microscope 2 and the object,
a reference position of the surgical microscope 2 (reference focal position=200 mm, reference zoom factor of the observation optical unit gamma_observation=1, reference stop position: stop 100% open),
a reference brightness of the illumination of 50%: reference age of the lamp: new xenon lamp,
a reference setting of the illumination stop (completely open) and illumination zoom (mid position of the illumination zoom, zoom factor gamma_illumination=1),
a reference setting of the image sensor parameters: gain=0, exposure time 20 ms, etc.

The calibration under the reference conditions leads to the determination of the signal at the image sensor 23 or image sensors 61A, 61B (i.e., the central reference pixel of the image sensor in the present example) which is generated by the light reflected by the first reference object if the illumination of the first reference object is implemented at the reference brightness and the remaining reference settings are present. By way of example, the first reference object generates a raw reference signal of 100 HE (brightness units) on the reference pixel of the image sensor 23 or image sensors 61A, 61B as a result of reflecting the illumination light under the aforementioned reference conditions. Thus, for the case of the reference conditions being present, a reference intensity in the form of a reference signal of likewise 100 HE is stored in the data processing unit.

Moreover, the calibration under the reference conditions leads to the determination of the signal at the image sensor 23 or image sensors 61A, 61B (i.e., the central reference pixel of the image sensor in the present example) which is present if the fluorescence is implemented in the second reference object with the reference intensity of the excitation wavelength. By way of example, the second reference object generates a raw reference signal of 100 HE (brightness units) on the reference pixel of the image sensor 23 or image sensors 61A, 61B as a result of a fluorescence emission excited by the excitation wavelength under reference conditions. Thus, for the case of the reference conditions being present, a reference intensity in the form of a reference signal of likewise 100 HE is stored in the data processing unit.

The object of the calibration is to ensure that, independently of whether the actual parameters values deviate from the reference parameter values, the same signal of 100 HE is always detected by the optical observation system 100 according to the invention when the reference object is observed. It is a further object of the calibration that there is a linear relationship between the diffuse reflectivity of an observation object 3 and the acquired signal, or between the fluorescent dye concentration of the observation object 3 and the acquired signal. By way of example, in the case of a fluorescence observation with a concentration of fluorescent dye of 10 nM, 50 nM, 70 nM, etc., which deviates from the reference concentration of 100 nM, a corrected signal of 10 HE, 50 HE, 70 HE, etc., should be detected in the case of an otherwise identical second reference object. For the case of an observation of reflected light (e.g., white-light observation), for example, a first reference object with a diffuse reflectivity of 10%, 20%, 30%, etc., which deviates from the reference reflectivity of 50%, should always be detected with a corrected signal of 10 HE, 20 HE, 30 HE, etc.

To ensure this, the data processing unit 6 of the present exemplary embodiment stores, in the memory of the signal processing unit by way of calibration or calculation in advance and for each parameter which influences the observation of the fluorescence intensity, a relationship between the deviation of the actual value of the respective parameter from its reference value and the deviation of the signal at the image sensor 23 or image sensors 61A, 61B which represents the measured intensity from the reference signal which represents the reference intensity. By way of example, a data processing unit 6 may store the fact that an actual zoom factor gamma_observation=2, which deviates from the reference zoom factor gamma_observation=1 of the observation optical unit, leads to a reduction in the signal, and hence in the measured intensity, by a factor of 0.25. By way of example, the relationship may be stored as an equation or a system of equations. Alternatively, it may be stored in the form of a value table or a plurality of value tables. It is also possible that the relationship is stored as an equation or system of equations for some parameters and stored in the form of a value table or a plurality of value tables for other parameters. Interpolations or extrapolations may also be carried out between the stored values in the case of one or more value tables.

With the aid of the formula or formulas stored in the data processing unit 6 or with the aid of the value table or value tables stored in the data processing unit 6, it is possible to convert a measured intensity, in particular a measured fluorescence intensity, into a corrected intensity, in particular a corrected fluorescence intensity. By way of example, if only the actual zoom factor gamma_observation=2 deviates from the reference zoom factor gamma_observation=1 in the case of actual conditions of the observation which otherwise correspond to the reference conditions, then the image sensor 23 or image sensors 61A, 61B capture a measured raw signal of 25 HE for the reference concentration of fluorescent dye. Thus, to correct the raw signal, the raw signal is divided by a deviation factor of 0.25 in order to obtain a corrected signal of 100 HE, which corresponds to the fluorescence intensity of the reference concentration of the fluorescent dye of 100 nM.

In another example, the raw signal has been reduced for example by a deviation factor of 0.2 on account of vignetting of the optical unit at the edge of the image sensor 23 or image sensors 61A, 61B, in the case of otherwise unchanged reference conditions. To correct the raw signal, the latter is divided by the deviation factor of 0.2 for pixels at the edge of the image sensor 23 or image sensors 61A, 61B, with the result that a corrected signal of 100 HE arises in turn.

In yet another example, the raw signal has been increased by a factor of 9 to a raw signal of 900 HE in the case of an illumination zoom factor gamma_illumination=3 and otherwise unchanged reference conditions. Thus, to correct the raw signal, the raw signal of 900 HE is divided by a deviation factor of 9 in order to obtain a corrected signal of 100 HE, which corresponds to the fluorescence intensity of the reference concentration of the fluorescent dye of 100 nM.

Various parameters which influence the observation of the fluorescence intensity may also be coupled to one another; for example, the vignetting may depend both on the location of the image sensor 23 or image sensors 61A, 61B and on the optical observation parameters of zoom, focus, and stop position. In this case, appropriately linked value tables or formulas are stored for the deviation factors.

In general, the correction method must be carried out separately for each pixel of the image sensor 23 or image sensors 61A, 61B, since some deviation factors depend on the location of the image sensor 23 or image sensors 61A, 61B (vignetting, for example). However, a majority of the deviation factors are independent of the location of the image sensor 23 or image sensors 61A, 61B (e.g., intensity setting of the light source), with the result that these represent global deviation factors for all locations on the image sensor 23 or image sensors 61A, 61B.

As a result of the above-described procedure, the reference object with 100 nM concentration of the fluorescent dye always has a corrected measurement value of 100 HE, and an actual object whose only deviation from the reference object is the concentration of the fluorescent dye of 33 nM always has a corrected measurement value of 33 HE, etc. Consequently, if all current parameter values of the parameters which influence the observation of the fluorescence intensity are known and if the respective arising deviation factors are stored correctly in the system, then the corrected signal depends only on the properties of the observation object 3 itself (e.g., optical properties, concentration of the fluorescent dye in the object, . . . ) and not on the settings of the surgical microscope 2 or the geometric position of the observation object 3 relative to the surgical microscope 2.

In a real system, it is not possible to determine and suitably correct all current parameter values of the parameter which influence the observation of the fluorescence intensity. However, it is possible to at least compensate the influences of as many of the parameters which influence the observation of the fluorescence intensity as possible, with the result that the corrected signal is (virtually) independent of at least the current parameter values of these parameters. In order nevertheless to obtain a reliable corrected signal, the user of the surgical microscope 2 should ensure that all parameters which influence the observation of the fluorescence intensity and which are not correctable by the system correspond to the reference conditions. For example, if the influence of the illumination intensity on the signal is not stored in correctable fashion in the system, then the user should ensure that the reference illumination intensity is set.

Naturally, the calibration may also be carried out if the simulation is only intended to determine, for each object region, the fluorescence intensity expected with the minimum concentration.

Fluorescent dyes such as PPIX slowly bleach upon excitation and successively lose fluorescence intensity. Therefore, the data processing unit 6 of a development of the optical observation system 100 depicted in FIG. 1 preferably registers, for each object region 3A-H of the observation object 3, the duration for which the corresponding object region 3A-H has already been illuminated by the excitation wavelength. The fluorescence signal emanating from the respective object region 3A-H is then corrected in each case by means of the data processing unit 6 by way of a bleaching factor that is determined on the basis of the duration, or else an alert is displayed if the duration and intensity of the illumination with the excitation wavelength have exceeded a level considered compatible with the fluorescent dye. A tracking system and the determination of a depth map are preferably provided in the case of a movement of the surgical microscope 2 relative to the observation object 3 in order to be able to assign the respective object regions 3A-H to the previous object regions 3A-H with the correct location in the new perspective. As it were, each object region 3A-H of the observation object 3 is provided with a counter which counts the amount of excitation light already radiated onto this point.

The present invention has been described in detail on the basis of exemplary embodiments for explanatory purposes. However, a person skilled in the art recognizes that there may be deviations from the described exemplary embodiments within the scope of the invention. Therefore, the invention is not intended to be limited by the exemplary embodiments but rather only consists of the appended claims.

1. A method for preparing the observation of a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100), using which fluorescence radiation is able to be observed, provided that this has a certain minimum intensity, and wherein the method comprises the following steps:
determining the parameter value of at least one parameter which influences the observation of the fluorescence intensity, and
simulating the fluorescence intensity expected for the respective object regions (3A-H) on the basis of the determined parameter value of the at least one parameter and a model of the influence of the at least one parameter on the fluorescence intensity,
wherein
a minimum concentration of the fluorescent dye is predefined within the scope of the simulation and
the fluorescence intensity expected with the minimum concentration is determined for each object region (3A-H) based on the simulation.

2. The method as claimed in claim 1, wherein a check is carried out, for each object region, as to whether the determined expected fluorescence intensity is sufficient to be able to be detected by the optical observation system (100) with the given sensitivity thereof.

3. The method as claimed in claim 1, wherein information about the depth distribution of the object regions (3A-H) and/or information about the orientation of the object regions (3A-H) is used within the scope of the simulation.

4. The method as claimed in claim 1, wherein at least the parameter value of one of the following parameters is determined and taken into consideration in the simulation:
distance of an optical observation device (2) of the observation system (100) from the object regions (3A-H),
orientation of the optical observation device (2) in relation to the object regions (3A-H),
zoom setting of the optical observation device (2),
front focal distance of the optical observation device (2),
stop setting of the optical observation device (2),
gain of an image sensor (23, 61A, 61B) used in the optical observation device (2),
exposure duration of an image sensor (23, 61A, 61B) used in the optical observation device (2),
nonlinearities of an image sensor (23, 61A, 61B) used in the optical observation device (2),
distance of an illumination system (40) from the object regions (3A-H),
orientation of the illumination system (40) of the observation system (100) in relation to the object regions (3A-H),
intensity of an illumination light source (41) of the illumination system (40);
spectral intensity distribution of an illumination light source (41),
zoom setting of an illumination zoom,
position of an illumination stop.

5. The method as claimed in claim 4, wherein the spectral intensity distribution of the illumination light source (41) is determined based on the value of a service life counter of the illumination source (41) and its nominally set intensity using a degradation model of the illumination source (41).

6. The method as claimed in claim 1, wherein an alert is output when the check reveals that the expected fluorescence intensity determined for the minimum concentration of the fluorescent dye is not sufficient, in each object region (3A-H), to be able to be detected by the optical observation system (100) with the given sensitivity thereof.

7. The method as claimed in claim 1, wherein a graphical display is generated, which displays the object regions (3A-H) in which the determined expected fluorescence intensity is sufficient to be able to be detected by the optical observation system (100) with the given sensitivity thereof, and the object regions (3A-H) in which it is not.

8. The method as claimed in claim 1, wherein, based on the simulation, an improved parameter value for the at least one parameter is determined such that the expected fluorescence intensity simulated with the improved parameter value is sufficient, in as many object regions (3A-H) as possible, to be detected by the optical observation system (100) with the given sensitivity thereof.

9. The method as claimed in claim 8, wherein the current parameter value of the at least one parameter is set automatically to the improved parameter value.

10. The method as claimed in claim 1, wherein
at least one reference measurement with a reference concentration of the fluorescent dye is carried out using a reference parameter value for the at least one parameter which influences the observation of the fluorescence intensity, in order to obtain a reference value for the fluorescence intensity at the reference concentration of the fluorescent dye,
a simulation of the fluorescence intensity expected for the respective object regions (3A-H) is carried out, in which a change in the fluorescence intensity in comparison to the reference intensity is determined for a deviation of the parameter value of the at least one parameter which influences the observation of the fluorescence intensity from the reference parameter value,
and a compensation factor is determined, by means of which it is possible to compensate a change in the fluorescence intensity in a digital image which is caused by the deviation of the parameter value of the at least one parameter which influences the observation of the fluorescence intensity from the reference parameter value.

11. A method for observing a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100), using which fluorescence radiation is able to be observed, provided that this has a certain minimum intensity, wherein it comprises the method for preparing the observation of a fluorescence intensity of fluorescence radiation of a fluorescent dye in an observation object (3) as claimed in claim 1.

12. An optical observation system (100) for observing a fluorescence intensity of fluorescence radiation of a fluorescent dye in an observation object (3) that comprises object regions (3A-H) that differ from one another in terms of their depth and/or their orientation, wherein the optical observation system (100) is designed such that it enables the observation of the fluorescence intensity, provided that this has a certain minimum intensity, and which comprises:
a determination device for determining the parameter value of at least one parameter which influences the observation of the fluorescence intensity, and
a simulation device (16) for simulating the fluorescence intensity expected for the respective object regions (3A-H) on the basis of the determined parameter value of the at least one parameter and a model of the influence of the at least one parameter on the fluorescence intensity,
characterized by an evaluation device (18) that is designed, for a minimum concentration of the fluorescent dye that is predefined within the scope of the simulation, to determine the fluorescence intensity expected with the minimum concentration for each object region (3A-H) based on the simulation.

13. The optical observation system (100) as claimed in claim 12, wherein the evaluation device (18) is designed to carry out a check, for each object region (3A-H), as to whether the determined expected fluorescence intensity is sufficient to be able to be detected by the optical observation system (100) with the given sensitivity thereof.

14. The optical observation system (100) as claimed in claim 12, wherein the simulation device (16) is designed to use information about the depth distribution of the object regions (3A-H) and/or information about the orientation of the object regions (3A-H) within the scope of the simulation.

15. The optical observation system (100) as claimed in claim 12, wherein the determination device (14) is designed to determine at least the parameter value of one of the following parameters and the simulation device (16) is designed to take this at least one parameter value into consideration in the simulation:
distance of an optical observation device (2) of the optical observation system (100) from the object regions (3A-H),
orientation of the optical observation device (2) in relation to the object regions,
zoom setting of the optical observation device (2),
front focal distance of the optical observation device (2),
stop setting of the optical observation device (2),
gain of an image sensor (23, 61A, 61B) used in the optical observation device (2),
exposure duration of an image sensor (23, 61A, 61B) used in the optical observation device (2),
nonlinearities of an image sensor (23, 61A, 61B) used in the optical observation device (2),
distance of an illumination system (40) of the optical observation system (100) from the object regions (3A-H),
orientation of the illumination system (40) in relation to the object regions (3A-H),
intensity of an illumination light source (41);
spectral intensity distribution of an illumination light source (41),
zoom setting of an illumination zoom,
position of an illumination stop.

16. The optical observation system (100) as claimed in claim 12, wherein the evaluation unit (18) is designed to generate a graphical display that displays the object regions (3A-H) in which the determined expected fluorescence intensity is sufficient to be able to be detected by the optical observation system (100) with the given sensitivity thereof.

17. The optical observation system (100) as claimed in claim 12, characterized by an optimization unit (22) that is designed, based on the simulation, to determine an improved parameter value for the at least one parameter such that the expected fluorescence intensity simulated with the improved parameter value is sufficient, in as many object regions (3A-H) as possible, to be detected by the optical observation system (100) with the given sensitivity thereof.

18. The optical observation system (100) as claimed in claim 17, characterized by a control unit (4) for controlling the optical observation system (100), which control unit is connected to the optimization unit (22) in order to receive the improved parameter value and is designed to set the at least one parameter to the improved parameter value.

19. The optical observation system (100) as claimed in claim 12, characterized by a compensation factor determination unit (24) that is designed to determine a compensation factor by way of which, in a digital image recorded by an image sensor (23, 61A, 61B), it is possible to compensate for a change in the fluorescence intensity caused by a deviation of the parameter value of the at least one parameter which influences the observation of the fluorescence intensity from a reference parameter value, wherein the compensation factor determination unit (24) is furthermore designed to determine the compensation factor on the basis of at least one reference value for the fluorescence intensity as determined for a reference concentration of the fluorescent dye and for a reference parameter value and of a simulation of the fluorescence intensity expected for the respective object regions, wherein, in the simulation, a change in the fluorescence intensity in comparison to the reference intensity is determined for a deviation of the parameter value of the at least one parameter which influences the observation of the fluorescence intensity from the reference parameter value.

20. A computer-implemented method for preparing the observation of a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100), using which fluorescence radiation is able to be observed, provided that this has a certain minimum intensity, and wherein the method comprises the following steps:
receiving or retrieving the parameter value of at least one parameter which influences the observation of the fluorescence intensity, and
simulating the fluorescence intensity expected for the respective object regions (3A-H) on the basis of the determined parameter value of the at least one parameter and a model of the influence of the at least one parameter on the fluorescence intensity,
wherein
a minimum concentration of the fluorescent dye is predefined within the scope of the simulation and
the fluorescence intensity expected with the minimum concentration is determined for each object region (3A-H) based on the simulation.

21. A computer program for preparing the observation of a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100), using which fluorescence radiation is able to be observed, provided that this has a certain minimum intensity, wherein the computer program comprises instructions which, when executed on a computer, prompt the latter to:
receive or retrieve the parameter value of at least one parameter which influences the observation of the fluorescence intensity, and
simulate the fluorescence intensity expected for the respective object regions (3A-H) on the basis of the received or retrieved parameter value of the at least one parameter and a model of the influence of the at least one parameter on the fluorescence intensity,
wherein
a minimum concentration of the fluorescent dye is predefined within the scope of the simulation and
the fluorescence intensity expected with the minimum concentration is determined for each object region (3A-H) based on the simulation.

22. A data processing unit (6) for preparing the observation of a fluorescence intensity of fluorescence radiation of a fluorescent dye in an observation object (3) that comprises object regions (3A-H) that differ from one another in terms of their depth and/or their orientation, wherein the observation is intended to be implemented using an optical observation system (100), using which fluorescence radiation is able to be observed, provided that this has a certain minimum intensity, wherein the data processing unit (6) comprises a memory and a processor and the processor is designed, by means of a computer program stored in the memory, to:
receive or retrieve the parameter value of at least one parameter which influences the observation of the fluorescence intensity, and
simulate the fluorescence intensity expected for the respective object regions (3A-H) on the basis of the received or retrieved parameter value of the at least one parameter and a model of the influence of the at least one parameter on the fluorescence intensity,
wherein
a minimum concentration of the fluorescent dye is predefined within the scope of the simulation and
the fluorescence intensity expected with the minimum concentration is determined for each object region (3A-H) based on the simulation.
